SEQUENCE LISTING (12 sequences)

SEQ ID NO: 1 - DNA, 2019 bp
  sequence encoding synthetic fusion protein
tggagccacccgcagttcgaaaaaatcgaagggcgccatcaccatcaccatcacggggcccagccggccatggcggactacaaagatattgtgatgacccaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgctcacgttcggtgctgggaccaagctggaaatcaaacgtggtggtggtggttctggtggtggtggttctggcggcggcggctccagtggtggtggatcccaggttcagcttcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtcaccgtctcctcggcctcgggggccggtggtggcggcagtggtggtggcggcagtggtggtggcggcagtggtggtggcggcagtgcggccgcgctagagggcggcagcctggccgcgctgaccgcgcaccaggcctgccacctgccgctggagactttcacccgtcatcgccagccgcgcggctgggaacaactggagcagtgcggctatccggtgcagcggctggtcgccctctacctggcggcgcgactgtcatggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccagcgccgacgtggtgagcctgacctgcccggtcgccgccggtgaatgcgcgggcccggcggacagcggcgacgccctgctggagcgcaactatcccactggcgcggagttcctcggcgacggtggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggatctcgacgcgatctggcgcggtttctatatcgccggcgatccggcgctggcctacggctacgcccaggaccaggaacccgacgcgcgcggccggatccgcaacggtgccctgctgcgggtctatgtgccgcgctcgagcctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgaaggacgagctg

SEQ ID NO: 2 - protein, 673 aa, Artificial Sequence
  synthetic fusion protein
WSHPQFEKIEGRHHHHHHGAQPAMADYKDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPLTFGAGTKLEIKRGGGGSGGGGSGGGGSSGGGSQVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVTVSSASGAGGGGSGGGGSGGGGSGGGGSAAALEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 3 - protein, 361 aa, Artificial Sequence
  modified exotoxin A sequence
EGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 4 - protein, 20 aa, Artificial Sequence
  synthetic linker
GGGGSGGGGSGGGGSSGGGS

SEQ ID NO: 5 - protein, 20 aa, Artificial Sequence
  synthetic linker
GGGGSGGGGSGGGGSGGGGS

SEQ ID NO: 6 - protein, 4 aa, Artificial Sequence
  synthetic transport sequence
KDEL

SEQ ID NO: 7 - protein, 5 aa, Artificial Sequence
  synthetic transport sequence
REDLK

SEQ ID NO: 8 - protein, 8 aa, Artificial Sequence
  synthetic strep tag
WSHPQFEK

SEQ ID NO: 9 - protein, 113 aa, Artificial Sequence
  4G7chim Variable light chain
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPLTFGAGTKLEIRR

SEQ ID NO: 10 - protein, 123 aa, Artificial Sequence
  4G7chim Variable heavy chain
  variation (118): Xaa = Thr or Ser
  variation (119): Xaa = Val or Leu or Ile or Met
QVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLXXTVSS

SEQ ID NO: 11 - DNA, 339 bp, Artificial Sequence
  sequence encoding 4G7chim light chain
gatattgtgatgacccaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgctcacgttcggtgctgggaccaagcttgagatcagacga

SEQ ID NO: 12 - DNA, 369 bp, Artificial Sequence
  sequence encoding 4G7chim heavy chain
caggttcagcttcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtcaccgtctcctcg

(19) United States
(12) Patent Application Publication
(10) Pub. No.: US 2009/0220501 A1
(43) Pub. Date: Sep. 3, 2009

(54) Anti-CD19 Antibody, Immunotoxin and Treatment Method

(76) Inventors: Georg H. Fey, Neunkirchen a. Br. (DE); Matthias Peipp, Hamburg (DE); Michael Schwemmlein, Erlangen (DE); Bruce Wang, Mountain View, CA (US); Matthias Wabl, San Francisco, CA (US)

Correspondence Address: King & Spalding LLP, P.O. Box 889, Belmont, CA, 94002-0889, US

(21) Appl. No.: 12/175,368

(22) Filed: Jul. 17, 2008

Related U.S. Application Data
(63) Continuation-in-part of application No. 11/344,466, filed on Jan. 30, 2006, now abandoned.
(60) Provisional application No. 60/954,998, filed on Aug. 9, 2007.

(51) Int. Cl. A61K 39/395; C07K 16/28

(52) U.S. Cl. 424/133.1; 424/179.1; 424/183.1; 530/387.3; 530/391.3; 530/391.7; 530/391.9

(57) ABSTRACT

Provided is an immunotoxin including (a) an anti-CD19 antibody lacking an Fc fragment, (b) a modified exotoxin A protein having both Domains II and III, but lacking Domain I, and (c) a peptide linker joining the C-terminal end of the antibody to the N-terminal end of the modified exotoxin A protein. The linker is substantially resistant to extracellular cleavage. The modified exotoxin A protein may be further modified to include a C-terminal KDEL sequence (SEQ ID NO: 6) that promotes transport of the protein to the endoplasmic reticulum of cells that have taken up the immunotoxin. Also provided is an anti-CD19 antibody having enhanced binding activity, antibody-dependent cellular cytotoxicity (ADCC) and methods for using the antibody to treat a disease state associated with B-lineage cells that express CD19. The antibody variable light and variable heavy chains have unique sequences in their J region relative to known anti-CD19 antibody sequences.

### CROSS REFERENCE TO RELATED APPLICATIONS

This application is a continuation-in-part, claiming benefit of priority under 35 U.S.C. § 120 to application Ser. No. 11/344,466, filed 30 Jan. 2006. This application also claims benefit under 35 U.S.C. § 119(e) to provisional application Ser. No. 60/954,998, filed 9 Aug. 2007, the contents of each of which are incorporated herein by reference.

### FIELD OF THE DISCLOSURE

The present disclosure relates to a humanized anti-CD19-specific antibody with enhanced binding affinity, ADCC activity for therapeutic uses, and to a method of making and using the antibody. Also provided are an immunotoxin and treatment methods employing the immunotoxin.

### BACKGROUND

CD19, a cell surface glycoprotein of the immunoglobulin superfamily is a potentially attractive target for antibody therapy of B-lymphoid malignancies. This antigen is absent from hematopoietic stem cells, and in healthy individuals its presence is exclusively restricted to the B-lineage and possibly some follicular dendritic cells (Scheuermann, R. et al. (1995) Leuk Lymphoma 18, 385-397). Furthermore, CD19 is not shed from the cell surface and is rarely lost during neoplastic transformation (Scheuermann, 1995). The protein is expressed on most malignant B-lineage cells, including cells from patients with chronic lymphocytic leukemia (CLL), Non-Hodgkin lymphoma (NHL), and acute lymphoblastic leukemia (ALL) (Uckun, F. M. et al. (1988) Blood 71, 13-29). Importantly, CD19 is consistently expressed on B-precursor and mature B-ALLs, whereas CD20 is less frequently expressed, particularly on B-precursor ALLs (Hoelzer, D. et al. (2002) Hematology (Am Soc Hematol Educ Program), 162-192). Therefore, only a portion of these patients can be treated with CD20 antibodies. In contrast, the majority of these patients might benefit from treatment with CD19-specific antibodies, if suitable antibodies were available.

Immunotoxins composed of a toxin linked to an antibody specific against cell-surface antigens, including CD19, have been proposed in the treatment of various cancers. However, such immunoconjugates have been limited in their use, heretofore, by extracellular cytotoxicity problems, such as hepatotoxicity, pulmonary toxicity, and/or severe hypersensitivity reactions. Ideally, an immunotoxin for use in treating B-cell malignancies would have a reduced toxicity before being taken up into target cells, and efficient uptake and retention within target cells. A therapeutic anti-CD19 antibody would ideally (i) be a humanized antibody, (ii) have an enhanced binding affinity constant, and (iii) efficiently promote antibody-dependent cell-mediated cytotoxicity (ADCC) of CD19-expressing cells in the presence of effector cells. The present disclosure is aimed at providing such an immunotoxin and antibody, and their use in treating disease states associated with B-lineage cells that express CD19, such as cancer and autoimmune conditions.

### SUMMARY

The present disclosure includes, in one aspect, a humanized anti-CD19 antibody characterized by (i) a dissociation constant Kd of about 5.5±1.7 nM or lower, as measured by a flow cytometry-based method where CD19-positive cells are first incubated with varying concentrations of anti-CD19 antibody, then incubated with a fluorescently labeled secondary antibody, followed by flow cytometry analysis to detect cell-bound anti-CD19 antibody; (ii) an ability to promote antigen-dependent cellular cytotoxicity, as measured by quantifying the amount of cell lysis of CD19-positive target cells such as acute lymphoblastic leukemia (ALL) cells after incubation with effector NK cells from healthy people and with the anti-CD19 antibody, and (iii) a variable heavy-chain sequence identified by SEQ ID NO:10, or a variable light-chain sequence identified by SEQ ID NO:9, or both sequences.

The antibody may be produced, for example, by recombinant baculovirus production in insect cells or by recombinant expression in a mammalian system, e.g., a Chinese Hamster Ovary (CHO) cell line, and in particular, in a mammalian system, such as CHO cells, in which the antibody is expressed in the presence of a beta-(1,4)-N-acetylglucosaminyltransferase III (GnTIII) enzyme.

Also disclosed is an antibody conjugate comprising the above anti-CD19 antibody covalently linked to a therapeutic moiety, such as a chemotherapeutic agent, a radiotherapy agent, a radiochemical therapeutic agent, or a toxin. The antibody or antibody conjugate may be formulated in an aqueous pharmaceutical carrier as a pharmaceutical composition.

The present disclosure includes, in one aspect, an immunotoxin for use in treating a subject having a cancer associated with malignant B-lineage cells, such as chronic lymphocytic leukemia, Non-Hodgkin lymphoma, and acute lymphoblastic leukemia. The immunotoxin includes (a) a anti-CD19 antibody lacking an Fc fragment, (b) a modified Pseudomonas aeruginosa exotoxin A protein having both Domains II and III, but lacking Domain I, and (c) a peptide linker joining the C-terminal end of the antibody to the N-terminal end of the modified exotoxin A protein. The linker is substantially resistant to extracellular cleavage.

The exotoxin A protein may have a C-terminal KDEL sequence (SEQ ID NO: 6) that promotes transport of the protein to the endoplasmic reticulum of cells that have taken up the immunotoxin, such as the modified exotoxin A protein having the sequence identified by SEQ ID NO: 3.

The antibody may be a single-chain scFv antibody composed of a variable-region light chain coupled to a variable-region heavy chain through a glycine/serine-peptide linker.

The antibody may be coupled to the modified exotoxin A protein through a glycine/serine-peptide linker, such as the linker having the sequence identified as SEQ ID NO: 5.

The antibody, for example, the antibody alone, may act as a growth arrest signal when it binds to CD19-positive cells. The antibody, for example, the antibody in the form of an antibody-toxin conjugate, may be internalized to arrest cell growth.

In another aspect, the disclosure provides a method for treating a subject having a disease state associated with B-lineage cells that express CD19, by administering to the subject a therapeutic amount of the above anti-CD19 antibody. In another aspect, the disclosure provides a method for treating a subject having a cancer associated with malignant B-lineage cells, such as chronic lymphocytic leukemia, Non-Hodgkin lymphoma, and acute lymphoblastic leukemia. The method comprises administering to the patient, a therapeutically effective amount of the above immunotoxin.

In still another aspect, the present disclosure includes a method for treating an autoimmune disease, such as multiple sclerosis, rheumatoid arthritis, and SLE, comprising administering to the patient, a therapeutically effective amount of the above immunotoxin.

Also disclosed is a method for delivering exotoxin A (ETA) to a human subject, in the treatment of a cancer having cancer-specific cell-surface antigens. The method comprises (a) replacing Domain I of the ETA with a single-chain antibody specific against the cell-surface antigen and a peptide linker joining the C-terminal end of the antibody to the N-terminal end of the modified ETA, and (b) replacing the REDLK C-terminal sequence (SEQ ID NO: 7) of ETA with a KDEL sequence (SEQ ID NO: 6) that promotes transport of the protein to the endoplasmic reticulum. The linker is substantially resistant to extracellular cleavage.

For use in treating a subject having a cancer associated with malignant B-lineage cells, such as chronic lymphocytic leukemia, Non-Hodgkin lymphoma, and acute lymphoblastic leukemia, the single-chain antibody replacing the ETA Domain I may be an antibody specific against CD19 B-cell antigen, such as an anti-CD19 scFv antibody. The linker may include a glycine/serine-peptide linker, such as a linker having the sequence identified as SEQ ID NO: 5.

Where the disease state is a cancer, such as B-cell subtype non-Hodgkin's lymphoma (NHL); Burkitt's lymphoma; multiple myeloma; pre-B acute lymphoblastic leukemia, acute lymphocytic leukemia; chronic lymphocytic leukemia; hairy cell leukemia; Null-acute lymphoblastic leukemia; Waldenstrom's Macroglobulinemia; and pro-lymphocytic leukemia; plasmacytoma; osteosclerotic myeloma; plasma cell leukemia; monoclonal gammopathy of undetermined significance (MGUS); smoldering multiple myeloma (SMM); indolent multiple myeloma (IMM); or Hodgkin's lymphoma, the CD19 antibody may be administered in an amount between 300 and 500 mg/m², with at least four separate doses separated by at least 7 days between doses.

For use in treating a subject having a B-lineage leukemia, where the subject is initially treated by transplantation of positive-selected stem cells to the patient, the CD19-antibody may be administered 7 to 14 days following the transplantation, in an amount effective to remove residual B-lineage leukemia cells from the patient.

For use in treating a subject having an autoimmune disease state associated with B-lineage cells that express CD19, such as rheumatoid arthritis, multiple sclerosis, and myasthenia gravis, the CD19 antibody may be administered in an amount between 300 and 500 mg/m², with at least two separate doses separated by at least 7 days between doses.

These and other objects and features of the disclosure will become more fully apparent when the following detailed description is read in conjunction with the accompanying drawings.

### BRIEF DESCRIPTION OF THE DRAWINGS

FIG. 1 is a schematic representation of the recombinant immunotoxin CD19-ETA′. STREP, N-terminal STREP tag; 6×His, hexahistidine tag; V_{L} and V_{H}, variable region light and heavy chains of the CD19-specific scFv; linker, flexible linkers consisting of glycine and serine residues; Exotoxin A′, truncated Exotoxin A fragment consisting of domains II and III of the Pseudomonas toxin; KDEL (SEQ ID NO: 6), ER retention motif.

FIGS. 2A and 2B are each a graph of the number of cells versus the fluorescence intensity showing specific binding of the recombinant immunotoxin to antigen-positive cells. Cells were stained with purified CD19-ETA′ fusion protein (black) or a nonrelated scFv-ETA′ fusion protein (white) at the same concentration and analyzed by FACS. FIG. 2A shows results for CD19-positive Namalwa cells stained with CD19-ETA′. FIG. 2B shows results for CD19-negative U937 cells stained with CD19-ETA′.

FIG. 3 is a graph showing the results of how CD19-ETA′ reduces the number of viable Nalm-6 cells during 96 hrs. Nalm-6 cells were treated with PBS or CD19-ETA′ at time point 0. At the indicated time points, viable cells were counted by trypan blue exclusion. Triplicate samples were measured for each time point and standard deviations are indicated by error bars.

FIGS. 4A and 4B are graphs showing the results of how CD19-ETA′ induces cell death of CD19-positive Nalm-6 cells at low concentrations but not of CD19-negative CEM cells. Nalm-6 (FIG. 4A) and CEM cells (FIG. 4B) were treated with single doses of the indicated concentrations of CD19-ETA′ for 72 h. Aliquots of cells were evaluated for percentage of cell death by PI staining of nuclei and FACS analysis. Data points are mean values from four independent experiments and standard deviations are indicated by error bars.

FIG. 5 shows images of cells stained with Annexin V and PI after 48 h of treatment with CD19-ETA′. The results show that CD19-ETA′ induces apoptosis in CD19-positive Nalm-6 (frames A-C), Namalwa (frames D-F) and Reh cells (frames G-I). Preincubation of the cells with the parental antibody 4G7 prevents the cells from being killed by CD19-ETA′. The cells were treated with PBS alone (frames A, D. and G), single doses of 500 ng/ml CD19-ETA′ alone (frames B, E, and H) or were preincubated with a molar excess of the parental CD19 antibody 4G7 (frames C, F, and I). Numbers in the upper right quadrant of each plot represent the percentage of Annexin V-positive cells.

FIGS. 6A and 6B are graphs showing the results of how CD19-ETA′ kills primary cells of two patients suffering from chronic lymphocytic leukemia (CLL) (6A and 6B). Primary CLL cells were treated with PBS (white bars), CD19-ETA′ (black bars) or a control immunotoxin CD33-ETA′ (grey bars) at time point 0. At the indicated time points, the percentage of Annexin V-positive cells was determined. Triplicate samples were measured for each time point and standard deviations are indicated by error bars.

FIGS. 7A and 7B show the coding and corresponding amino acid sequences of the variable light chain (SEQ ID NO: 11) and variable heavy chain (SEQ ID NO: 12) of the anti-CD19 antibody of the present disclosure;

FIGS. 8A and 8B show amino-acid sequence alignment between the variable light chain (8A) and variable heavy chain (8B) from the known 4G7 anti-CD19 antibody (upper line) and the anti-CD19 antibody of the present disclosure (lower line);

FIG. 9 plots the percent cell lysis of primary B-lineage ALL blast cells in the presence of (i) healthy donor NK cells alone (open triangles); (ii) NK cells plus the anti-CD19 antibody of the present disclosure (closed triangles); (iii) NK cells induced with IL-2 (open squares), and (iv) NK cells induced with IL-2 plus the anti-CD19 antibody of the present disclosure (closed squares), all at four different effector cell-target cell ratios;

FIG. 10 shows percent cell lysis of primary B-lineage ALL blast cells in the presence of healthy donor NK cells alone plus (i) the anti-CD19 antibody of the present disclosure, (ii) no added antibody, (iii) α-HLA-1 antibody, (iv) α-HLA-1 Fab antibody fragment, (v) murine anti-CD19 antibody, and (vi) anti-CD20 antibody;

FIGS. 11A and 11B show ADCC against MHH4 target cells with donor-derived mononuclear cells (MNCs) obtained from patients after T-cell depleted stem cell transplantation, where the ADCC effect is shown with a patient Group I (11A), and a patient Group 11 (11B), classified according to the strength of the ADCC response with the patient donor cells;

FIG. 12 shows percent ADCC against MHH4 target cells with donor-derived MNCs, comparing the ADCC effect of the anti-CD19 antibody of the present disclosure against an anti-CD20 antibody;

FIG. 13 plots the percent cell lysis of primary B-lineage ALL blast cells in the presence of (i) donor MNC alone (open triangles); (ii) donor MNC plus the anti-CD19 antibody of the present disclosure (closed triangles); (iii) donor MNC induced with IL-2 (open squares), and (iv) donor MMC induced with IL-2 plus the anti-CD19 antibody of the present disclosure (closed squares), all at four different effector cell-target cell ratios; and

FIG. 14 shows percent cell lysis of primary B-lineage ALL blast cells in the presence of donor MNC healthy donor NK cells alone plus (i) α-HLA-1 antibody, (ii) no antibody, (iii) the anti-CD19 antibody of the present disclosure, (iv) murine anti-CD19 antibody, and (v) anti-CD20 antibody.

### DETAILED DESCRIPTION

### I. Definitions

The following terms have the meaning defined herein, except when indicated otherwise.

An “anti-CD19 antibody” or “CD19-specific antibody” or “CD19 antibody” refers to an antibody that specifically recognizes the cell-surface glycoprotein of the immunoglobulin superfamily commonly referred to as CD19.

The term “antibody”, as used herein, encompasses immunoglobulin molecules comprised of four polypeptide chains, two heavy (H) chains and two light (L) chains inter-connected by disulfide bonds. Each chain consists of a variable portion, denoted V_{H} and V_{L} for variable heavy and variable light portions, respectively, and a constant region, denoted CH and CL for constant heavy and constant light portions, respectively. The CH portion contains three domains CH1, CH2, and CH3. Each variable portion is composed of three hypervariable complementarity determining regions (CDRs) and four framework regions (FRs). The region between the each variable region and constant region in a light chain is known as a junction region; in heavy chains the variable and constant regions are separated by a diversity region and a junction region.

The Fc fragment of an antibody refers to the crystalline fragment of an immunoglobulin which is released by, e.g., papain digestion of an immunoglobulin, and which is responsible for many of the effector functions of immunoglobulins.

The two heavy chains in an antibody form an Fc region that mediates effector functions, such as antibody-dependent cellular cytotoxicity (ADCC) and complement-dependent cytotoxicity (CDC). In ADCC, the Fc region of an antibody binds to Fc receptors (FcyRs) on the surface of immune effector cells such as natural killers and mononuclear cells, leading to the phagocytosis or lysis of the targeted cells. In CDC, the antibodies kill the targeted cells by triggering the complement cascade at the cell surface. IgG isoforms exert different levels of effector functions increasing in the order of IgG4<IgG2<IgG1≦IgG3. Human IgG1 displays high ADCC and CDC, and is the most preferred isoform for antibodies of the present disclosure.

An “antibody lacking an Fc fragment” refers to any of a variety of antibody fragments lacking the effector functions of the Fc fragment, and may include (i) an Fab fragment, which is a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). In particular, although the two domains of the Fv fragment, V_{L} and V_{H}, are coded for by separate genes, they can be joined by recombinant methods, by a synthetic linker that enables them to be made as a single protein chain in which the V_{L} and V_{H} regions pair to form monovalent molecules known as single chain variable fragment or scFv antibodies; see e.g., Bird et al. (1988) Science 242:423-426; and Huston et al. (1988) Proc. Natl. Acad. Sci. USA 85:5879-5883), and the term antibody lacking an Fc fragment also encompasses antibodies having this scFv format.

A “humanized anti-CD19 antibody” or “chimeric humanized antibody” refers to an anti-CD19 antibody derived from a non-human antibody, typically a murine anti-CD19 antibody, that retains or substantially retains the variable-light and/or variable-heavy chain sequences of the non-human antibody, but where some or all constant regions of the antibody have been replaced by human antibody sequences. A humanized antibody is typically a “recombinant antibody,” meaning that the antibody is prepared, expressed, created or isolated by recombinant means, such as antibodies expressed using a recombinant expression vector transfected into a host cell.

A “glycine/serine” linker refers to a linear polypeptide chain composed substantially, e.g., at least 80%, and preferably entirely of glycine and serine amino acid residues.

“GnTIII” enzyme refers to beta-(1,4)-N-acetylglucosaminyltransferase III, as described, for example, in U.S. Pat. No. 6,602,684.

The three-letter and one-letter amino acid abbreviations and the single-letter nucleotide base abbreviations used herein are according to established convention, as given in any standard biochemistry or molecular biology textbook.

### II. Anti-CD19 Antibody Preparation and Construction of the Anti-CD19 Immunotoxin

This section describes the preparation and characterization of a humanized anti-CD19 antibody in accordance with the present disclosure, that is, a humanized anti-CD19 antibody characterized by:

(i) a dissociation constant K_{d} of about 5.5±1.7 nM or lower (lower K_{d}, meaning higher binding affinity), as measured by a flow cytometry-based method where CD19-positive cells are first incubated with varying concentrations of anti-CD19 antibody, then incubated with a fluorescently labeled secondary antibody, followed by flow cytometry analysis to detect cell-bound anti-CD19 antibody;

(ii) an ability to promote antigen-dependent cellular cytotoxicity, as measured by quantifying the amount of cell lysis of CD19-positive target cells such as acute lymphoblastic leukemia cells after incubation with effector cells such as NK cells from healthy people and with the anti-CD19 antibody, and

(iii) a variable heavy-chain sequence identified by amino-acid SEQ ID NO:10 or a light-chain sequence identified by amino-acid SEQ ID NO: 9, or both variable heavy- and variable light-chain sequences identified by SEQ ID NOS: 10 and 9, respectively.

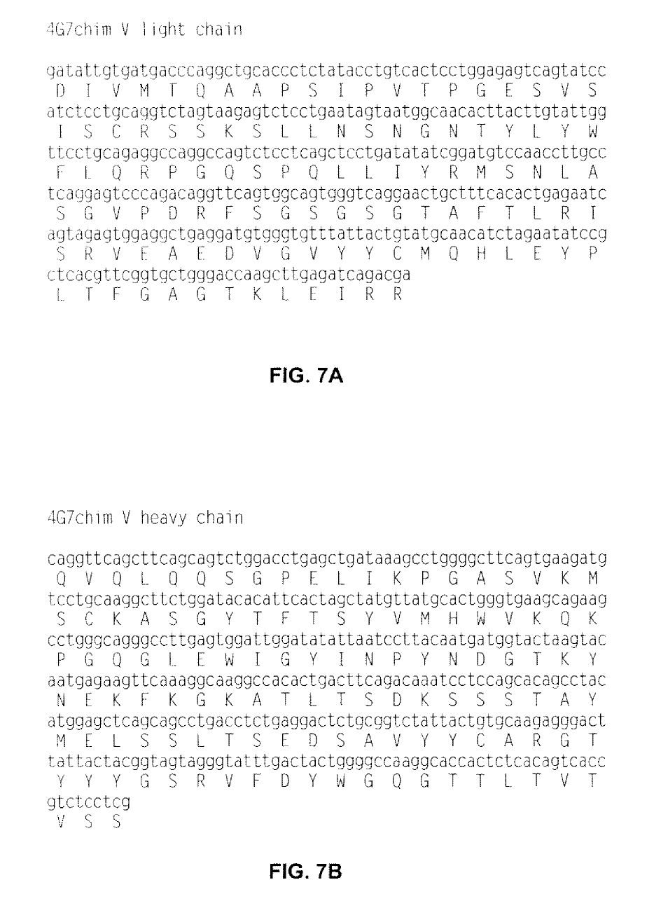

Amino acid sequences of the variable light and heavy chains. The amino acid sequence of the variable light-chain sequence identified as SEQ ID NO: 9 is given in FIG. 7A, along with the corresponding coding sequence, identified as SEQ ID NO: 11. The amino acid sequence of the variable heavy-chain sequence identified as SEQ ID NO: 10 is shown in FIG. 7B, along with the corresponding coding sequence, identified as SEQ ID NO: 12. Note that SEQ ID. 10 includes conservative substitutions (T and S) and (V, L, I, and M) at residue positions 118 and 119, respectively, in the J region of the variable region adjacent the C-terminal end of the sequence.

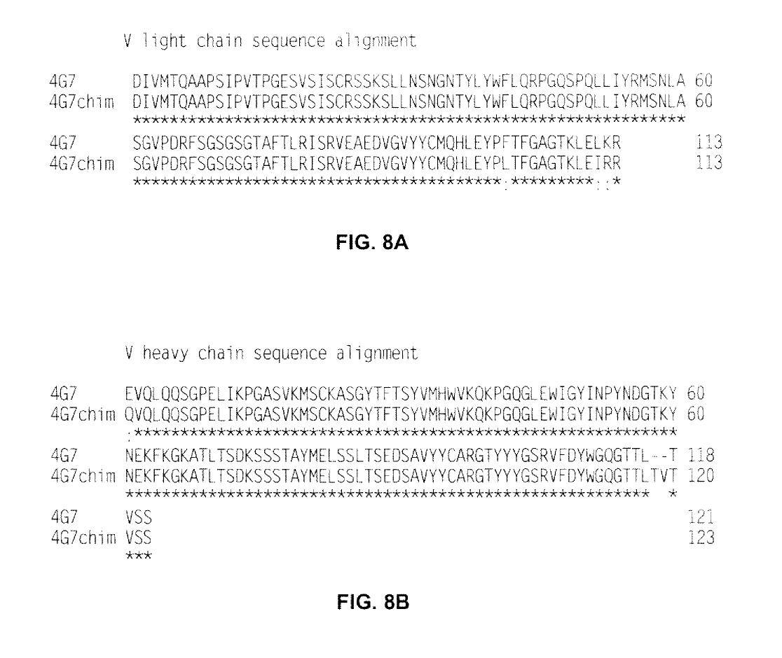

The variable light and heavy chain sequences from FIGS. 7A and 7B, respectively, were compared by sequence alignment with the corresponding variable light (GenBank ID AJ555479) and heavy chain (GenBank ID AJ555622) sequences from the parent 4G7 murine anti-CD19 antibody from which the present antibody was derived. The sequence alignment between variable light chain sequences in FIG. 8A shows substitutions at three amino acid residues, positions 101, 111, and 112. Of these, the more meaningful variations may be the conservative amino acid substitutions at positions 111 and 112 in the J region of the light-chain variable region.

The sequence alignment between the variable heavy chain sequences, shown in FIG. 8B, shows a substitution at position 1 and an addition of two amino acids at positions 118, 119, in the J region of the sequence adjacent the C-terminal end of the region. As noted above, SEQ ID NO: 10 for the variable heavy-chain sequence includes neutral amino acid substitutions at both of these positions. In one embodiment, the human anti-CD19 antibody of the disclosure includes the variable heavy chain sequence identified as SEQ ID NO: 10, but where at least one of T and V residues at positions 118 and 119, respectively, has been substituted by one of the indicated conservative amino acid substitution, that is, S for T and/or L, I, or M for V.

Preparation of humanized anti-CD19 antibody. Humanized antibodies can be prepared based on the sequence of a murine monoclonal antibody prepared according to conventional monoclonal antibody techniques. DNA encoding the heavy and light chain immunoglobulins can be obtained from the murine hybridoma of interest and engineered to contain human immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

More generally, humanized antibodies may be prepared by (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

The present disclosure also includes an immunotoxin composed of (1) a CD19-specific antibody lacking an Fc fragment, e.g., a single chain Fv (scFV) antibody fragment, (2) an engineered variant of Pseudomonas Exotoxin A (ETA) having both Domains II and III, but lacking Domain I, and (3) a peptide linker joining the C-terminal end of the antibody to the N-terminal end of the modified exotoxin A protein. The linker is substantially resistant to extracellular cleavage.

A CD19-specific antibody lacking an Fc fragment may be constructed according to known methods. Where the antibody is an anti-CD19 scFv antibody, the methods detailed in Example 1 are suitable. In one exemplary method, variable heavy- and variable light-chain sequences were amplified by polymerase chain reaction (PCR), using degenerate-sequence primers, from the DNA of CD19-reactive scFvs previously generated from the hybridoma 4G7 (Meeker T C, Miller R A, Link M P, et al., A unique human B lymphocyte antigen defined by a monoclonal antibody. Hybridoma. (1984) 3:305-320) by the phage display technique using standard procedures (e.g., Krebber A, Bornhauser S, Burmester J, et al. Reliable cloning of functional antibody variable domains from hybridomas and spleen cell repertoires employing a reengineered phage display system. J Immunol Methods. (1997) 201: 35-55; and Peipp M, Kupers H, Saul D, et al. A recombinant CD7-specific single-chain immunotoxin is a potent inducer of apoptosis in acute leukemic T cells. Cancer Res. (2002) 62: 2848-2855). Cleavage sites for restriction enzymes were introduced and subsequently used for the insertion of these fragments into the baculoviral expression vector pAc-K-CH3 (Liang M, Dubel S, Li D, et al. Baculovirus expression cassette vectors for rapid production of complete human IgG from phage display selected antibody fragments. J Immunol Methods. (2001) 247: 119-130). Sf21 insect cells were cotransfected with the baculoviral expression construct and Sapphire Baculovirus DNA (Orbigen, San Diego, Calif.). Purification of the secreted recombinant protein from culture supernatants was performed by protein A agarose affinity chromatography.

As just noted, the toxin moiety of the immunotoxin is Pseudomonas Exotoxin A (ETA), specifically, a truncated version lacking domain I and containing only domains II and III. (Wels, W., Beerli, R., Hellmann, P., Schmidt, M., Marte, B. M., Kornilova, E. S., Hekele, A., Mendelsohn, J., Groner, B., and Hynes, N. E). The EGF receptor and p185erbB-2-specific single-chain antibody toxins differ in their cell-killing activity on tumor cells expressing both receptor proteins. Int J Cancer, 60: 137-144, 1995). Domain I is the binding domain for the α₂-macroglobulin receptor (CD91) present on most mammalian cells (Kounnas, M. Z., Morris, R. E., Thompson, M. R., FitzGerald, D. J., Strickland, D. K., and Saelinger, C. B. The alpha 2-macroglobulin receptor/low density lipoprotein receptor-related protein binds and internalizes Pseudomonas exotoxin A. J Biol Chem, 267: 12420-12423,1992).

Domains II and III of ETA are required for intracellular transport and carry the active center of the toxin, respectively, which inhibits protein synthesis by blocking the translation elongation factor EF-2 and causes apoptosis (Lord, J. M., Smith, D. C., and Roberts, L. M. Toxin entry: how bacterial proteins get into mammalian cells. Cell Microbiol, 1: 85-91,1999). Consequently, the truncated variant of ETA, abbreviated ETA′, which lacks domain I is not toxic as long as it remains in the extracellular space. In addition, ETA′ can be administered with fewer side effects on vascular endothelial cells, because it has a much lower affinity to these cells than, for example, ricin A.

Replacing the domain I of ETA with an antibody fragment directed against an antigen capable of internalization, converts the ETA′ variant into a potent immunotoxin. Moreover, the modified ETA′ may be further modified to contain a C-terminal KDEL (SEQ ID NO: 6) motif, the characteristic ER retention sequence of a variety of luminal ER proteins (Munro, S. and Pelham, H. R. A C-terminal signal prevents secretion of luminal ER proteins. Cell, 48: 899-907,1987). Further, coupling the modified ETA to the CD-19 antibody through a linker that is substantially resistant to extracellular cleavage reduces the potential for toxicity due to release of the toxin into the bloodstream before the immunotoxin reaches the target cells. As will be seen below, the immunotoxin shows that a CD19-specific scFv fused to ETA′ is effective at very low concentrations against CD19-positive leukemia cell lines and primary cells from CLL patients, and displays exquisite antigen-specific activity.

To construct the coding sequence for the immunotoxin protein, the scFv cDNA insert from a reactive phage isolate was subcloned and fused to the coding sequence for truncated Pseudomonas Exotoxin A lacking the receptor-binding domain (Example 2). The coding sequence for the C-terminal pentapeptide REDLK (SEQ ID NO: 7), a peptide directing the retrograde transport of the authentic toxin, was replaced by the coding sequence for the KDEL-tetrapeptide (SEQ ID NO: 6), a peptide assuring proper retrograde transport of cellular proteins. This replacement was performed following published examples (Brinkmann, U., Pai, L. H., FitzGerald, D. J., Willingham, M., and Pastan, I. B3(Fv)-PE38KDEL, a single-chain immunotoxin that causes complete regression of a human carcinoma in mice. Proc Natl Acad Sci USA, 88: 8616-8620,1991) to optimize intracellular transport to the ER. In one embodiment, the variable light and heavy chain domains (V_{L} and V_{H}) are linked by a sequence coding for a 20 amino acid synthetic linker, and given by SEQ ID NO: 4. In the same embodiment, the scFv antibody and ETA′ toxin are linked by a sequence coding for a 20 amino acid synthetic linker, and given by SEQ ID NO: 5.

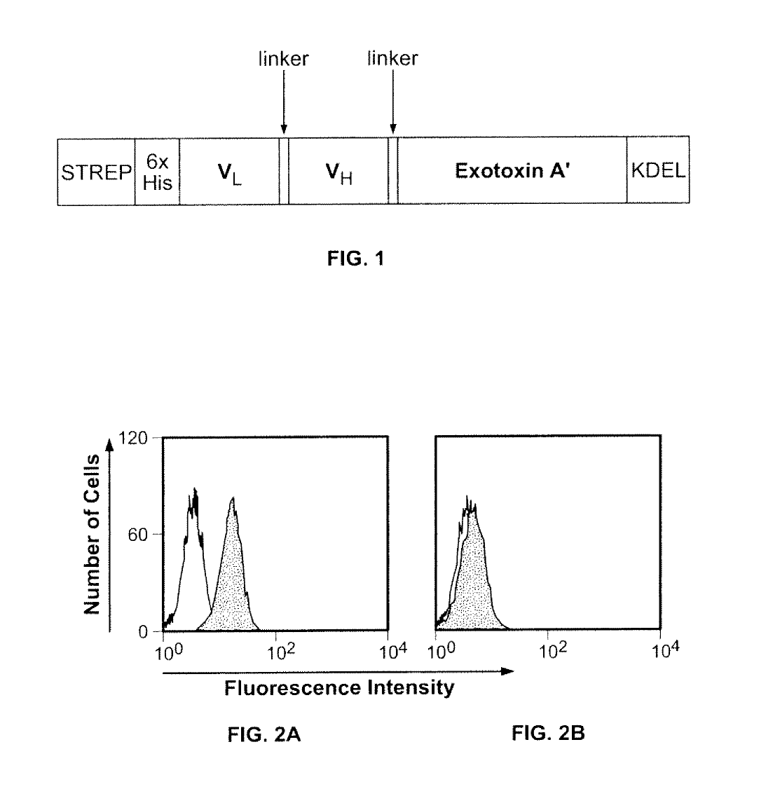

Sequences coding for a STREP-tag (WSHPQFEK, SEQ ID NO: 8) and a hexahistidine-tag were added at the N-terminus for detection and purification and a schematic representation of the resulting purified fusion protein is shown in FIG. 1. The complete coding sequence for the fusion protein is given by SEQ ID NO:1 below, and the amino acids sequence for the fusion protein, by SEQ ID NO: 2. The resulting polypeptide was expressed in E. coli and purified from periplasmic extracts by affinity chromatography using a streptactin matrix. The fusion protein which is referred to as the CD19-immunotoxin (termed CD19-ETA′) specifically reacted with the CD19-positive human Burkitt lymphoma derived cell line Namalwa as visualized by flow cytometry (see FIG. 2). The agent failed to react with CD19-negative monocytic U937-cells.

In an alternative embodiment, the anti-CD19 antibody is prepared as above, but is expressed in a mammalian expression system, such as Chinese Hamster Ovary (CHO) cells with a suitable known expression vector. In one exemplary embodiment, the antibody is expressed in a mammalian expression system, such as CHO cells, in the presence of the GnTIII enzyme, yielding an antibody having an enhanced portion of bisected oligosaccharides in the antibody Fc region, as described in U.S. Pat. No. 6,602,684, thus enhancing ADCC activity. Both the chimeric humanized antibody and GnTIII enzyme may be co-expressed in a suitable, preferably mammalian cell line that has been transfected, e.g., co-transfected or transfected sequentially with expression vectors for both antibody and enzyme.

Exemplary methods for recombinant expression of the anti-CD19 antibody of the disclosure are given in Examples 4 and 5 below.

### III. Binding Affinity and ADCC Activity of the Anti-CD19 Antibody

Binding affinity. The binding properties of the anti-CD19 antibody of the disclosure was examined by flow cytometric analysis and binding equilibrium studies, employing the methods given in Examples 6A and 6B, respectively. Briefly, the dissociation constant was measured by a flow cytometry-based method in which CD19-positive cells were first incubated with varying concentrations of anti-CD19 antibody, then incubated with a fluorescently labeled secondary antibody, followed by flow cytometry analysis to detect cell-bound anti-CD19 antibody (Benedict C A, MacKrell A J, and Anderson W F. Determination of the binding affinity of an anti-CD34 single-chain antibody using a novel, flow cytometry based assay. J Immunol Methods. 1997 February 28;201(2):223-31). Following this method, a Kd of about 5.5±1.7 nM was measured.

Anti-CD19 antibodies constructed in accordance with the present disclosure therefore have a binding affinity constant K_{d}, as measured by the flow cytometry method above, of about 5.5±1.7 nM or lower, that is, a value lower than 5.5±1.7 nM, meaning a higher binding affinity. This K_{d} compares with a K_{d} of about 10 nM for the known CD19 4G7 murine antibody from which the present antibody was derived. While not wishing to be bound to particular structure/activity relationships, it would appear that the lower K_{d} observed for the present antibody (higher binding affinity) is due to one or more of the five amino acid variations between the present antibody and the parent 4G7 antibody in the variable light- and variable heavy-chain sequences as discussed above. Of these, the two-amino acid addition at positions 118, 119 in the variable heavy-chain J region may be especially important.

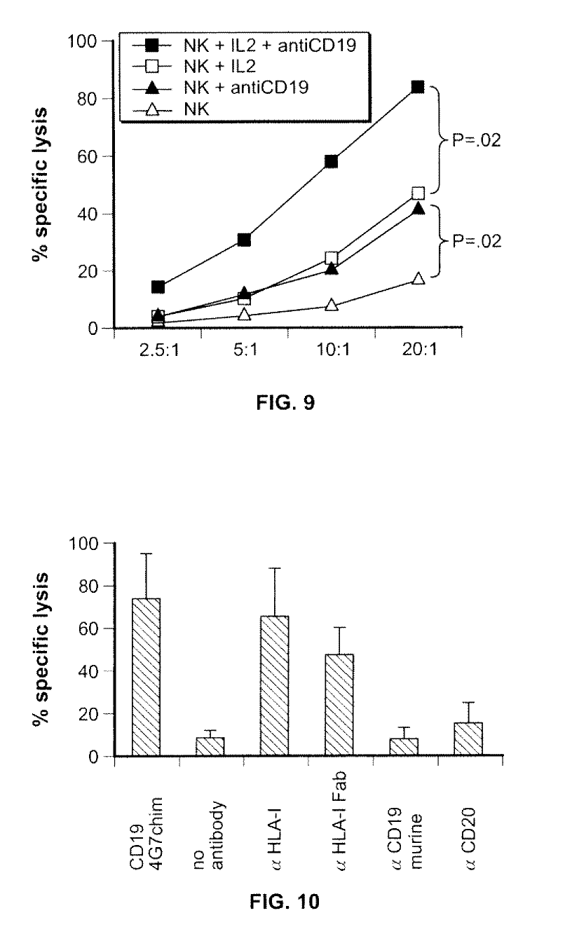

ADCC activity. The ability of the anti-CD19 antibody to promote antibody-dependent cell-mediated cytolysis (ADCC) of target cells, in the presence of suitable effector cells was examined by standard ADCC test procedures, as described in Example 7. In a first study, the target cells were cryopreserved primary B-lineage ALL blast cells from 3 different patients, and the effector cells were NK cells obtained from healthy donors. FIG. 9 plots the percent cell lysis in the presence of (i) healthy donor NK cells alone (open triangles); (ii) NK cells plus the anti-CD19 antibody of the disclosure (closed triangles); (iii) NK cells induced with IL-2 (open squares), and (iv) NK cells induced with IL-2 plus the anti-CD19 antibody of the disclosure (closed squares), all at the four different effector cell-target cell ratios indicated.

Considering the data from FIG. 9 and FIG. 10, the anti-CD19 antibody of the disclosure mediated specific lysis of cryopreserved primary pre-B/common ALL blasts with enriched NK cells from 7 healthy donors. NK cells from each donor showed significantly enhanced lysis after addition of the anti-CD19 antibody. Preincubation of the effector cells with IL-2 at 40 IU/mL increased specific lysis to its maximum value. Both these IL-2 and antibody concentrations fall into a range which may be reached in clinical applications. As expected, the murine 4G7 antibody did not induce ADCC. Specificity of anti-CD19 antibody was shown by addition of the chimeric CD20 IgG1 antibody, which failed to raise specific lysis.

Blocking of HLA class I antigens on target cells relevant for NK cell inhibitory receptors with complete W6/32 antibody or Fab fragments increased specific lysis comparable to the anti-CD19 antibody alone (FIG. 10), indicating that masking of HLA class I on ALL blasts may abolish possible HLA class I-mediated inhibition of NK cell lysis. However, these inhibitory effects did not prevent ADCC by the anti-CD19-antibody.

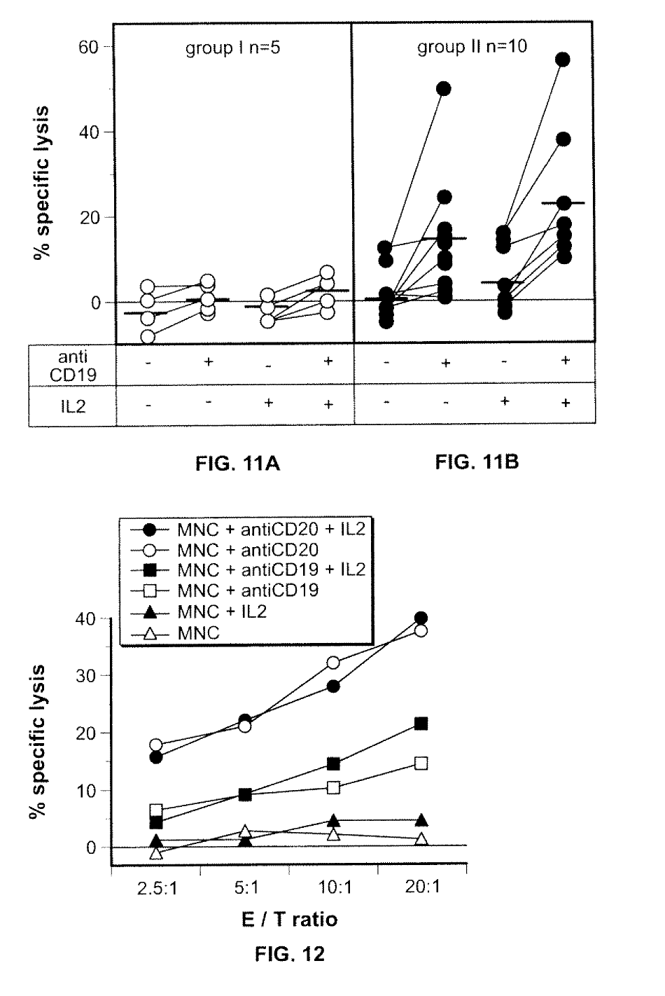

The ability of the anti-CD19 antibody to promote ADCC with patient-derived effector cells was also investigated, with the results shown in FIGS. 11-14. Summarizing the results, effector mononuclear cells (MNCs) from the majority of patients (66%) produced substantial lysis of target MHH4 cells in combination with the anti-CD19 antibody. Lysis was enhanced by incubation of effector cells with IL-2 (FIG. 11A). Although Mabthera™ was more effective than the anti-CD19 antibody against CD19⁺CD20⁺targets (FIG. 12), for the treatment of pediatric B-lineage leukemias, often lacking CD20 expression, a CD19-directed antibody therapy would be recommended. Effector cells from the other one third of patients produced specific lysis below 10%, although the percentage of NK cells among MNCs was not significantly different for both groups (FIG. 11B).

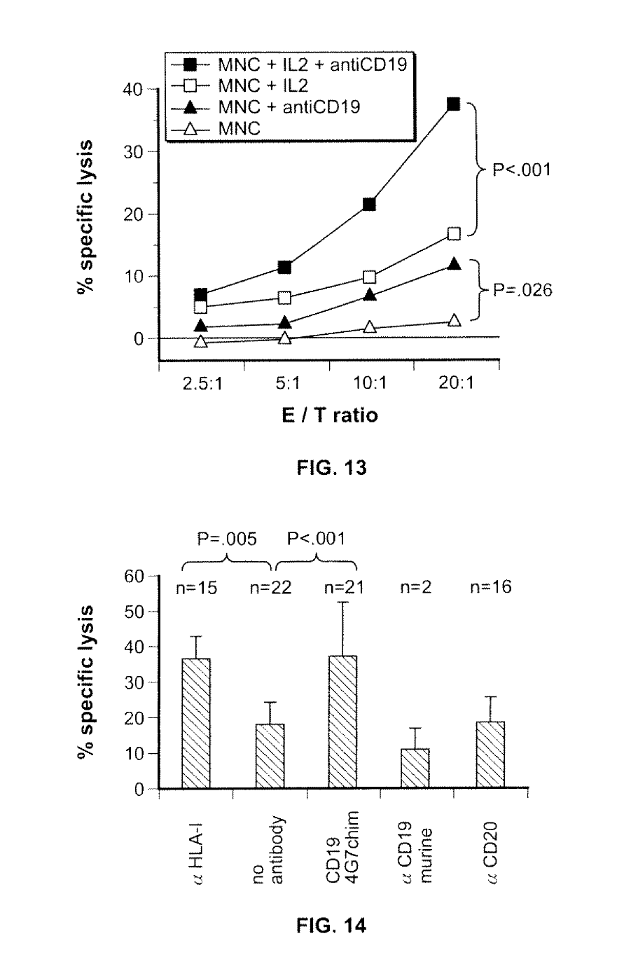

In contrast to MHH4 cells, cryopreserved primary B-lineage blasts were more susceptible to NK lysis, although leukemic blasts from a patient with refractory disease were predominantly used as targets. In repeated experiments with effector cells from 7 patients against these allogeneic blasts and from 1 patient for whom autologous blasts were available, a consistent pattern emerged: specific lysis by unstimulated MNCs was less extensive than specific lysis by unstimulated MNCs plus the anti-CD19 antibody. IL-2 stimulation of effector cells increased specific lysis. Combination of antibody and IL-2 stimulation significantly enhanced specific lysis to a maximum (FIG. 13). As expected, both the parental murine 4G7 antibody and Mabthera™, used as controls, failed to raise specific lysis, consistent with the fact that these target cells were CD20-(FIG. 14). Blocking of HLA class I with W6/32 antibody raised specific lysis to the same extent as treatment with the anti-CD19 antibody, suggesting that NK cells were the major effectors and that the anti-CD19 antibody mediated ADCC also in the posttransplantation setting, despite possible HLA class I-induced inhibition of effector cells. ALL patients in particular may benefit from such antibody-augmented GVL (graft-versus-leukemia) effects, because NK cell alloreactivity alone was shown to have no effect on adult ALL even in mismatched transplantations.

IV. Characterization of an scFv-ETA′ Immunotoxin

### A. Antigen-Specific Cytotoxic Activity of the Immunotoxin

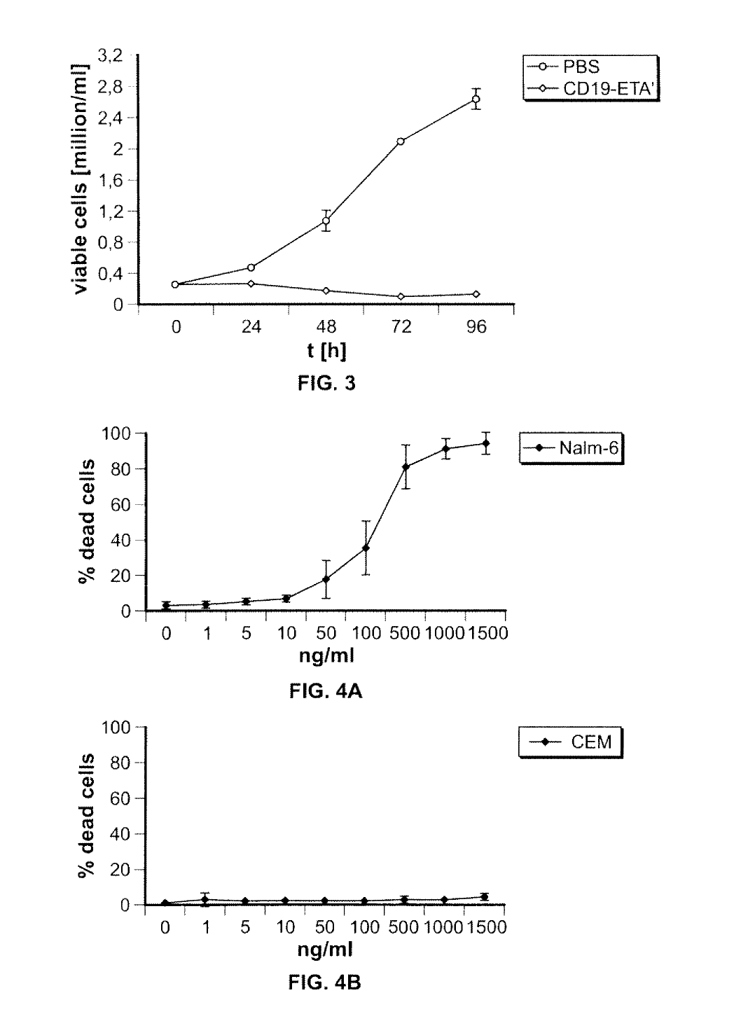

CD19-ETA′ mediated specific death of CD19-positive Nalm-6 cells, but failed to eliminate CD19-negative CEM cells, as evidenced by counting viable cells every 24 h for 96 h (FIG. 3), and measurement of nuclear DNA content after 72 h of treatment, using propidium iodide (PI) staining and flow cytometry with the results being graphed in FIG. 4. Maximum lysis of Nalm-6 cells within 72 h was achieved with single doses of 1 μg/ml (14 nM). Same concentrations of the immunotoxin failed to kill antigen-negative CEM cells. Thus, these results show that CD19-ETA′ acts in a highly antigen-specific manner and is effective for cultured malignant cells in the low nanomolar concentration range. The results demonstrate that the toxin is highly specific for cells expressing surface antigen CD19, and that selective cell killing is effective in the nM range of immunotoxin.

### B. CD19-ETA′ Eliminates Cells by Apoptosis.

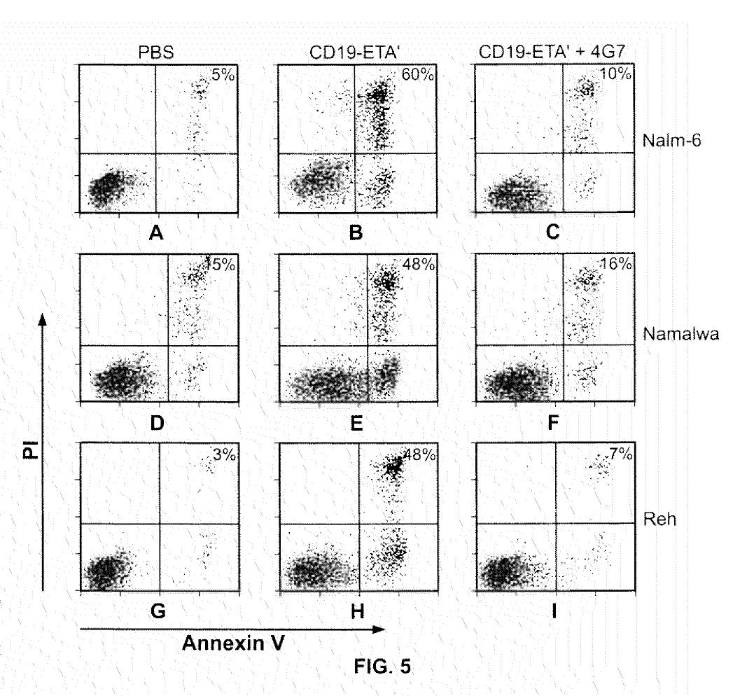

To investigate whether death induced by the agent occurred via apoptosis or other cellular routes to elimination, apoptosis was specifically measured by Annexin V and PI staining. This method of Annexin V and PI staining provides independent evidence for cell death by apoptosis beyond the method of counting cells with SubG₁-DNA content presented above (FIG. 4). CD19-ETA′ induced apoptosis of antigen-positive human B cell precursor leukemia derived cell lines Nalm-6 and Reh, and of human Burkitt lymphoma derived Namalwa cells. For comparison, cell death was blocked by pretreatment with excess concentrations of the parental CD19 antibody 4G7 (FIG. 5). These results confirm the ability of CD19-ETA′ to kill target cells by apoptosis in a highly antigen-specific manner for different CD19-positive tumor-derived human cell lines representing different disease entities.

### C. CD19-ETA′ Induces Cell Death of Primary CLL Cells

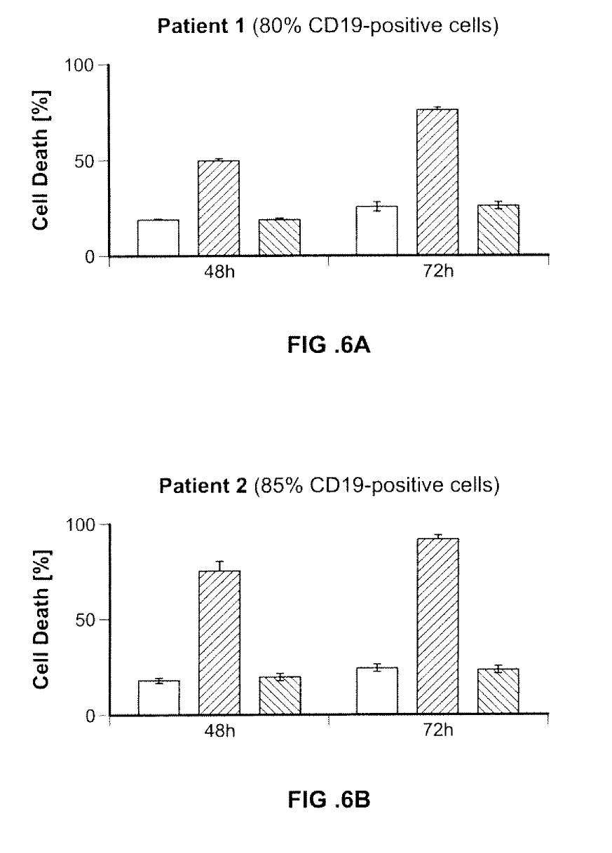

CD19-ETA′ also mediated death of primary cells from two patients suffering from CLL (FIG. 6). The induction of cell death by the CD19-ETA′ immunotoxin was antigen-specific because a control immunotoxin directed against an antigen not expressed on the CLL cells was not able to kill the cells.

### V. Therapeutic Method

The antibody and immunotoxin are useful in treating a human subject having a disease condition associated with B-lineage cells that express CD19, including malignancies associated with B-lineage cells, such as such as B-cell subtype non-Hodgkin's lymphoma (NHL); Burkitt's lymphoma; multiple myeloma; pre-B acute lymphoblastic leukemia, acute lymphocytic leukemia; chronic lymphocytic leukemia; hairy cell leukemia; Null-acute lymphoblastic leukaemia; Waldenstrom's Macroglobulinemia; pro-lymphocytic leukemia; plasmacytoma; osteosclerotic myeloma; plasma cell leukemia; monoclonal gammopathy of undetermined significance (MGUS); smoldering multiple myeloma (SMM); indolent multiple myeloma (IMM); or Hodgkin's lymphoma. In some embodiments, the therapeutic method is evidenced by (i) the ability of the immunotoxin to exhibit its cytotoxic effects in the concentration range of ng/ml, (ii) the cytolysis by the immunotoxin is highly antigen-specific, and (iii) immunotoxin induced cell death occurs by apoptosis as demonstrated by Annexin V staining.

In this immunotherapy approach, a patient diagnosed with a disease condition associated with B-lineage cells is treated by administration of the immunotoxin or anti-CD19 antibody. The antibody dose is preferably administered in an amount between 300 and 500 mg/m², with at least four doses separated by at least 7 days between doses. The antibody is administered by IV injection in a suitable physiological carrier. The immunotoxin dose is preferably 1 to 10 mg/injection, and the patient is treated at intervals of every 14 days or so.

For treating a subject having a B-lineage leukemia, wherein the subject is initially treated by transplantation of positive-selected stem cells to the patient, the CD19-antibody is administered 7 to 14 days following the transplantation, in an amount effective to remove residual B-lineage leukemia cells from the patient, e.g., 300 and 500 mg/m².

During treatment, the patient is monitored for change in status of the cancer, typically by standard blood cell assays. The treatment may be carried out in combination with other cancer treatments, including drug or radio-isotope therapy, and may be continued until a desired improvement in patient condition is attained.

The immunotoxin or anti-CD19 antibody is also useful in treating an autoimmune disease, such as multiple sclerosis, rheumatoid arthritis, and systemic lupus erythematosus (SLE). In this method, a patient diagnosed with an autoimmune disease is treated by administration of the immunotoxin or anti-CD19 antibody. Preferably the antibody is administered by IV injection in a suitable physiological carrier. The antibody dose is preferably 1 g to 2 g/injection, or about, 300 and 500 mg/m² and the patient is treated at intervals of approximately every 7-14 days. During treatment, the patient is monitored for improvement in status, e.g., reduced level of pain or discomfort associated with the condition. The treatment may be carried out in combination with other treatments, such as treatment with immunosuppressive drugs, and may be continued until a desired improvement in patient condition is attained, or over an extended period to alleviate symptoms.

As can be appreciated from the studies above, the immunotoxin and anti-CD19 antibody provide a number of advantages as therapeutic agents specific against CD-19 expressing cells. The immunotoxin and anti-CD19 antibody are highly specific against CD-19 expressing cells and are active at very low concentrations, e.g., in the nM range. Due to the absence of the Fc portion of the antibody in the immunotoxin, undesirable interactions of the Fc portion with Fc receptors on cells other than the tumor target cells are prevented. The stable link between antibody-portion and toxin moiety leads to reduced non-specific toxicities due to the breakage of this bond in the extracellular space, and ensures that the toxin will be largely confined to target cells.

The following examples illustrate, but are in no way intended to limit the present disclosure.

### Materials and methods

### A. Bacterial Strains and Plasmids

Escherichia coli XL1-Blue™ (Stratagene, Amsterdam, the Netherlands) was used for the amplification of plasmids and cloning, and E. coli TG1 (from Dr. G. Winter, MRC, Cambridge, United Kingdom) for screening of antibody libraries. Libraries were generated in the phagemid vector pAK100, and pAK400 was used for the expression of soluble scFvs (Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997). E. coli BL21 (DE3; Novagen, Inc., Madison, Wis.) served for the expression of scFv-ETA′ fusion protein.

B. Cell Lines

Leukemia-derived cell lines Nalm-6, Namalwa, Reh, CEM (DSMZ; German Collection of Microorganisms and Cell Lines, Braunschweig, Germany) and SEM (Greil, J., Gramatzki, M., Burger, R., Marschalek, R., Peltner, M., Trautmann, U., Hansen-Hagge, T. E. Bartram, C. E., Fey, G. H., Stehr, K. The acute lymphoblastic leukemia cell line SEM with t(4;11) chromosomal rearrangement is biphenotypic and responsive to interleukin-7. Br J Haematol, 86: 275-283, 1994) were cultured in RPMI 1640-Glutamax™-I (Sigma, Deisenhofen, Germany) containing 10% FCS and penicillin and streptomycin (Invitrogen) at 100 units/ml and 100 μg/ml, respectively.

Human 293T embryonal kidney cells, 293 cells and 293 cells stably expressing human CD19 were cultured in DMEM-Glutamax™-I medium (Invitrogen, Karlsruhe, Germany) supplemented with 10% fetal calf serum (FCS), 1% penicillin and streptomycin (Invitrogen). ARH-77 (EBV-transformed B-lymphoblastoid cell line established from a patient with plasma cell leukemia; from the American Type Culture Collection, ATCC) and SEM cells, derived from a patient with B-precursor ALL (Greil et al. (1994) Br J Haematol 86, 275-283), were cultured in RPMI 1640-Glutamax™-I medium (Invitrogen), containing 10% FCS, 1% penicillin and streptomycin (Invitrogen).

### EXAMPLE 1

### Preparation of CD-19 scFv Antibody

Total RNA was prepared from the hybridoma 4G7 (Meeker, T. C., Miller, R. A., Link, M. P., Bindl, J., Warnke, R., and Levy, R. A unique human B lymphocyte antigen defined by a monoclonal antibody, Hybridoma, 3: 305-320, 1984; Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997). First-strand cDNA was prepared from 10-15 μg of total RNA (Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997). PCR amplification of immunoglobulin variable region cDNAs and cloning into the phagemid vector pAK100 was performed as described (Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997; Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001). Propagation of combinatorial scFv libraries and filamentous phages was performed by following published procedures (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001).

A. Panning of Phage Display Libraries with Intact Cells

Panning of phage display libraries with intact cells was carried out as described (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001) using CD19-positive SEM cells. Bound phages were eluted with 50 mM HCl.

B. Bacterial Expression and Purification of Soluble scFv Antibodies

For the soluble expression of antibody fragments, cDNA coding for the CD19-specific scFv was subcloned into the expression vector pAK400, and the plasmids were propagated in E. coli HB2151 (from Dr. G. Winter; MRC, Cambridge, United Kingdom). Expression and purification of CD19-specific scFv antibodies was performed as described (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001).

### EXAMPLE 2

### Construction and Expression of scFv-ETA′ Fusion Protein

Sequences coding for the CD19-specific scFv were excised from the pAK400-anti CD19 expression construct and were cloned into the vector pASK/HisCD19ETA#3 (M. Peipp, unpublished data). The plasmid was digested with NcoI and NotI and ligated into the vector pet27b(+)-Strep-His-CD33-ETA′-KDEL (M. Schwemmlein, unpublished data), resulting in the vector pet27b(+)-STREP-His-CD19-ETA′-KDEL.

The scFv-ETA′ fusion protein was expressed under osmotic stress conditions as described (Barth, S., Huhn, M., Matthey, B., Tawadros, S., Schnell, R., Schinkothe, T., Diehl, V., and Engert, A. Ki-4(scFv)-ETA′, a new recombinant anti-CD30 immunotoxin with highly specific cytotoxic activity against disseminated Hodgkin tumors in SCID mice. Blood, 95: 3909-3914, 2000). Induced cultures were harvested 16-20 h after induction. The bacterial pellet from 1 liter culture was resuspended in 200 ml of periplasmatic extraction buffer [100 mM Tris, pH 8.0, 0.5 M sucrose, 1 mM EDTA] for 3 h at 4° C. The scFv-ETA′ fusion protein was enriched by affinity chromatography using streptactin agarose beads (IBA GmbH, Goettingen, Germany; Skerra, A. and Schmidt, T. G. Use of the Strep-Tag and streptavidin for detection and purification of recombinant proteins. Methods Enzymol, 326: 271-304, 2000) according to manufacturers instructions.

### EXAMPLE 3

### Characterization of scFv-ETA′ Immunotoxin

### A. Immunotoxin Binding to Cells

The binding of scFvs to cells was analyzed using a FACSCalibur™ FACS instrument and CellQuest™ software (Becton Dickinson, Mountain View, Calif.). Cells were stained with scFv antibodies as described (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001). A nonrelated scFv served as a control for background staining. Ten thousand events were collected for each sample, and analyses of whole cells were performed using appropriate scatter gates to exclude cellular debris and aggregates. To monitor binding of the scFv-ETA′ fusion protein, 5×10⁵ cells were incubated for 30 min on ice with 20 μl of the immunotoxin at a concentration of 5 μg/ml. A nonrelated immunotoxin served as a control for background staining. The cells were washed with PBA buffer [containing PBS, 0.1% BSA, and 7 mM Na-azide] and then incubated with 50 μl of a polyclonal rabbit anti-Pseudomonas ETA serum (Sigma) diluted 1:250 in PBA buffer. Cells were washed and incubated with fluorescein-iso-thiocyanate (FITC)-conjugated pig anti-rabbit-IgG (DAKO Diagnostica GmbH, Hamburg, Germany) for 30 min. After a final wash, cells were analyzed by FACS.

### B. Measurement of Cytotoxic Effects of Immunotoxins

For dose response experiments, cells were seeded at 2.5×10⁵/ml in 24-well plates, and immunotoxin was added at varying concentrations. Cell death was measured by staining nuclei with a hypotonic solution of PI as described (Dorrie, J., Gerauer, H., Wachter, Y., and Zunino, S. J.). Resveratrol induces extensive apoptosis by depolarizing mitochondrial membranes and activating caspase-9 in acute lymphoblastic leukemia cells. Cancer Res, 61: 4731-4739, 2001; Nicoletti, I., Migliorati, G., Pagliacci, M. C., Grignani, F., and Riccardi, C. A rapid and simple method for measuring thymocyte apoptosis by propidium iodide staining and flow cytometry. J Immunol Methods, 139: 271-279,1991). The extent of cell death was determined by measuring the fraction of nuclei with subdiploid DNA content. Fifteen thousand events were collected for each sample and analyzed for subdiploid nuclear DNA content. To determine whether cell death was attributable to apoptosis, cells were seeded at 2.5×10⁵/ml and treated with the immunotoxin. Whole cells were stained with FITC-conjugated Annexin V (Pharmingen, Heidelberg, Germany; Vermes, I., Haanen, C., Steffens-Nakken, H., and Reutelingsperger, C. A novel assay for apoptosis. Flow cytometric detection of phosphatidylserine expression on early apoptotic cells using fluorescein labelled Annexin V. J Immunol Methods, 184: 39-51, 1995) and PI in PBS according to the manufacturer's protocol. For blocking experiments, a 20-fold molar excess of the parental CD19 antibody 4G7 was added to the culture 1 h before adding the immunotoxin. For determination of viable cells, cells were stained by trypan blue and counted.

### EXAMPLE 4

### CD19Specific Antibody and GnTIII Expression Vectors

### A. Expression Vector for the Humanized CD19-Specific IgG1 Antibody.

For expression of the CD19 chimeric antibody in Sf21 insect cells, the baculoviral expression vector pAc-K-CH3/4G7chim-Sf21 was used. This vector contained a fusion construct coding for a human immunoglobulin heavy (H) chain secretion leader; the variable region of the murine CD19 antibody of the invention and the complete constant region of human gamma 1 heavy chain, framed by BamH I restrictions sites. The light (L) chain sequence, consisting of a human immunoglobulin L-chain secretion leader, the variable region of the murine CD19 antibody of the invention and a human kappa L-chain constant domain, was framed by Bgl II restriction sites. For expression in mammalian cells, the H- and L-chain coding sequences were excised from this vector and inserted into the mammalian expression vector pBud/CHO (unpublished data) derived from the vector pBud CE4.1 (Invitrogen). The coding sequence for the antibody L-chain including the leader was excised from the baculoviral vector by digestion with Bgl II and was then inserted into the BamH I restriction site. Subsequently, the H-chain of the chimeric antibody with the corresponding human H-chain leader was excised by BamH I digestion from the baculoviral vector and inserted into the Bgl II restriction site, resulting in the mammalian expression vector pBud/4G7chim.

Expression vector for GnTIII. For production of anti-CD19 antibody in presence of GnTIII (GnTIII), the cDNA sequence coding for rat GnTIII was obtained from rat liver polyA mRNA (BD Biosciences Clontech, Palo Alto, Calif., USA) by reverse transcription using standard procedures. The GnTIII coding sequence was amplified with the 5′ primer GnTIII for (5′-ACG TGC TAG CCA CCA TGA GAC-3′), containing an Nhe I restriction site, and the 3′primer GnTIII back (5′-ACG TTT CTA GAT GGC CCT CCG-3′), containing an Xba I restriction site. The GnTIII fragment is digested with Nhe I and Xba I and inserted into the Nhe I/Xba I digested vector pSecTag2HygroC-GFP+/anti-CD19 HD37 scFv (Peipp et al. (2004) J Immunol Methods 285, 265-280), thereby generating the expression vector pSecTag2HygroC/GnTIII. This vector enables the intracellular expression of GnTIII fused to a C-terminal myc-tag and hexa-histidine tag, added for detection of the recombinant protein.

### EXAMPLE 5

### Expression and Purification of Anti-CD19 Antibody

For mammalian expression of the anti-CD19 antibody, 293T cells were transiently transfected using the calcium phosphate method including the addition of 50 μM chloroquine to the transfection mix (Sambrook. J., and Russel. D. W. (2001) Molecular Cloning: A Laboratory Manual. 3 Ed., Cold Spring Harbor Laboratory Press, Cold Spring Harbor, N.Y.). Transfection was performed with a total of 20 μg plasmid DNA per 100 mm culture dish, containing either the cDNA coding for 4G7chim alone or in combination with cDNA for GnTIII. After 9 h, transfection medium was replaced by fresh medium. After 48 h medium was exchanged, and culture supernatants were collected for five consecutive days. Purification of the secreted antibodies from culture supernatants was performed by affinity chromatography with protein A agarose (Sigma-Aldrich, Taufkirchen, Germany) according to manufacturer's instructions. For oligosaccharide analyses, the chimeric antibodies produced in 293T cells were purified via protein A chromatography on a 1 ml HiTrap™ protein A column (Amersham, Freiburg, Germany), using a pH gradient elution with a 100 mM phosphate-citrate buffer (pH7 to pH3), to eliminate bovine IgG.

### A. Determination of Antibody Concentration by Sandwich-ELISA

Antibody concentrations were determined following published procedures. Briefly, dilutions of a standard human IgG antibody (Sigma) with defined concentrations and dilutions of the purified antibody were incubated in EIA/RIA microplates (Corning, Wiesbaden, Germany), previously coated with a rabbit anti-human kappa chain antibody (DakoCytomation, Hamburg, Germany). Bound antibody was detected with a horseradish peroxidase-conjugated goat anti-human Fc antibody (Sigma), following development using the ABTS reagent (Roche Diagnostics, Mannheim, Germany). A standard curve was generated and relative to this curve concentrations of purified protein samples were determined.

### B. Cell Lysates, SDS-PAGE and Western Blot Analysis

Two days after transfection with expression constructs for the anti-CD19 antibody, cells were washed once with PBS and cell lysates were prepared by resuspension of 5 million cells in 100 μl lysis buffer (50 mM Tris HCl pH8, 150 mM NaCl, 0.02% NaN₃, 1% TritonX, 0.1% SDS) containing Complete™ Mini proteinase inhibitor (Roche Diagnostics). After incubation on ice for 30 minutes and vortexing, cell debris were removed by centrifugation at 4° C. and protein concentrations were determined using Bradford Reagent (Sigma). SDS-PAGE under reducing conditions was performed according to standard procedures (Sambrook, supra). 4G7chim was detected with a horseradish peroxidase-coupled secondary antibody against human IgG heavy chains (Sigma), GnTIII with a penta-histidine antibody (Qiagen) and a horseradish peroxidase-conjugated secondary antibody according to manufacturer's protocols. Western blots were developed using enhanced chemiluminescence reagents (Amersham).

### EXAMPLE 6

### Characterization of Antibody Binding Properties

### A. Flow Cytometric Analysis

For immunofluorescence analysis of the chimeric antibody, cells were incubated with the purified recombinant protein (1 μg/ml) or human IgG (Sigma) as an isotype control for 30 min on ice. After washing with PBS containing 0.1% bovine serum albumin and 7 mM sodium azide, an FITC-conjugated anti-human IgG (Sigma) was used as secondary antibody. Flow cytometry was performed on a FACSCalibur™ instrument with CellQuest™ software (Becton Dickinson, Heidelberg, Germany). For each sample 1×10⁴ events were collected and analyses of whole cells were performed using appropriate scatter gates to exclude cellular debris and aggregates.

### B. Determination of Antibody Equilibrium Constants (K_{D})

K_{D} values were determined by flow cytometry using published procedures (Benedict, C. A. et al. (1997) J Immunol Methods 201, 223-231). Experiments were repeated 6 times and mean values are reported. Values and graphical analyses were generated using GraphPad Prism Software (GraphPad Software Inc., San Diego, Calif., USA).

### EXAMPLE 7

### Cytotoxicity Studies

### A. Isolation of Mononuclear Cells (MNCs)

Twenty ml of peripheral blood was obtained from healthy volunteers and MNCs were isolated as described (Elsasser, D. et al. (1996) Blood 87, 3803-3812).

Purity of MNCs was assessed by cytospin preparations and exceeded 95%. Viability of cells was>95% as tested by trypan blue exclusion.

### B. Cytotoxicity Experiments

ADCC assays with NK cells from healthy donors or MNC cells from patients as effector cells were performed by a 3 h ⁵¹Cr release assay as described (Elsasser, supra), using ARH-77 cells as targets. Cytotoxicity experiments with purified NK cells as effectors and cryopreserved primary common ALL (cALL) blasts as target cells were performed in a 2 h BATDA (bis (acetoxymethyl) 2,2′:6′,2″-terpyridine-6,6″-dicarboxylate) europium release assay as previously described (Lang P. et al. (2004) Blood 103, 3982-3985). All ADCC assays were performed in triplicates.

### C. Statistical Analyses

Group data are reported as mean values±standard error of the mean (SEM). Differences between groups were analyzed by paired (or, when appropriate, unpaired) Student's t-test.

Although the present disclosure has been described with respect to particular embodiments and applications, it will be appreciated that various changes and modifications may be made without departing from the present disclosure and invention as claimed.

### Description of Sequences Listed:

SEQ ID NO: 1, polynucleotide sequence encoding the antibody-toxin conjugate;

SEQ ID NO: 2, amino acid sequence of the antibody-toxin conjugate;

SEQ ID NO: 3, amino acid sequence of the modified ETA′ protein;

SEQ ID NO: 4, amino acid sequence of the linker coupling the variable-light and variable-heavy chains of the scFv antibody;

SEQ ID NO: 5, amino acid sequence of the linker coupling the scFv antibody to the modified ETA′ toxin;

SEQ ID NO: 6, sequence that promotes transport of a protein to the endoplasmic reticulum;

SEQ ID NO: 7, sequence that promotes transport of a protein to the endoplasmic reticulum; and

SEQ ID NO: 8, STREP tag.

SEQ ID NO: 9, amino acid sequence of the 4G7chim Variable light chain.

SEQ ID NO: 10, amino acid sequence of the 4G7chim Variable heavy chain.

SEQ ID NO: 11, polynucleotide sequence encoding 4G7chim light chain.

SEQ ID NO: 12, polynucleotide sequence encoding 4G7chim heavy chain.

1. A humanized anti-CD19 antibody characterized by:
(i) a dissociation constant K_{d} of about 5.5±1.7 nM or lower, as measured by a flow cytometry-based method where CD19-positive cells are first incubated with varying concentrations of anti-CD19 antibody, then incubated with a fluorescently labeled secondary antibody, followed by flow cytometry analysis to detect cell-bound anti-CD19 antibody;
(ii) an ability to promote antigen-dependent cellular cytotoxicity, as measured by quantifying the amount of cell lysis of CD19-positive target cells such as acute lymphoblastic leukemia cells after incubation with effector cells such as NK cells from healthy people and with the anti-CD19 antibody, and
(iii) a variable heavy-chain sequence identified by SEQ ID NO:10.

2. The anti-CD19 antibody of claim 1, having a variable light-chain sequence identified by SEQ ID NO:9.

3. The anti-CD19 antibody of claim 1, produced by baculovirus production in insect cells.

4. The anti-CD19 antibody of claim 1, produced by recombinant expression in mammalian cells in the presence of a beta-(1,4)-N-acetylglucosaminyltransferase III (GnTIII) enzyme.

5. A humanized anti-CD19 antibody characterized by:
(i) a dissociation constant K_{d} of about 5.5±1.7 nM or lower, as measured by a flow cytometry-based method where CD19-positive cells are first incubated with varying concentrations of anti-CD19 antibody, then incubated with a fluorescently labeled secondary antibody, followed by flow cytometry analysis to detect cell-bound anti-CD19 antibody;
(ii) an ability to promote antigen-dependent cellular cytotoxicity, as measured by quantifying the amount of cell lysis of CD19-positive target cells (e.g., acute lymphoblastic leukemia cells) after incubation with effector cells (e.g., NK cells) from healthy people and with the anti-CD19 antibody, and
(iii) a variable light-chain sequence identified by SEQ ID NO:9

6. The anti-CD19 antibody of claim 5, having a variable heavy-chain sequence identified by SEQ ID NO:10.

7. The anti-CD19 antibody of claim 5, produced by recombinant baculovirus production in insect cells.

8. The anti-CD19 antibody of claim 5, produced by recombinant expression in mammalian cells in the presence of a beta-(1,4)-N-acetylglucosaminyltransferase III (GnTIII) enzyme.

9. An antibody conjugate comprising the antibody of claims 1 or 5 covalently linked to a therapeutic moiety selected from the group consisting of a chemotherapeutic agent, a radiotherapy agent, a radiochemical therapeutic agent, and a toxin.

10. A pharmaceutical composition comprising the antibody of claims 1 or 5 in an aqueous pharmaceutical carrier.

11. A method for treating a subject having a disease state associated with B-lineage cells that express CD19, comprising
administering to the subject, a therapeutic amount of the anti-CD19 antibody of claims 1 or 5.

12. The method of claim 11, wherein the disease state is a malignancy such as such as B-cell subtype non-Hodgkin's lymphoma (NHL); Burkitt's lymphoma; multiple myeloma; pre-B acute lymphoblastic leukemia, acute lymphocytic leukemia; chronic lymphocytic leukemia; hairy cell leukemia; Null-acute lymphoblastic leukemia; Waldenstrom's Macroglobulinemia; and pro-lymphocytic leukemia; plasmacytoma; osteosclerotic myeloma; plasma cell leukemia; monoclonal gammopathy of undetermined significance (MGUS); smoldering multiple myeloma (SMM); indolent multiple myeloma (IMM); or Hodgkin's lymphoma, and said CD19 antibody is administered in an amount between 300 and 500 mg/m², with at least four doses separated by at least 7 days between doses.

13. The method of claim 11, for treating a subject having a B-lineage leukemia, wherein the subject is initially treated by transplantation of positive-selected stem cells to the patient, and the anti-CD19 antibody is administered 7 to 14 days following the transplantation, in an amount effective to remove residual B-lineage leukemia cells from the patient.

14. The method of claim 11, for treating a subject having an autoimmune disease state associated with B-lineage cells that express CD19, such as rheumatoid arthritis, multiple sclerosis, myasthenia gravis, and lupus erythematosus, wherein said anti-CD19 antibody is administered in an amount between one and two grams, with at least two doses separated by at least 14 days between doses.

15. In a method of treating a subject having a leukemia associated with malignant B-lineage cells that express CD19, by allogeneic transplantation of positive-selected stem cells to the patient, an improvement for removing residual B-lineage leukemia cells from the patient, comprising
administering to the patient, at a selected period following said allogenic transplantation, an amount of the anti-CD19 antibody of claims 1 or 5 effective to remove residual B-lineage leukemia cells from the patient.

16. The improvement of claim 15, wherein said anti-CD19 antibody is administered in an amount between 300 and 500 mg/m², and at a selected period between 7 and 14 days after said allogenic transplantation.

17. An immunotoxin for use in treating a subject having a cancer associated with malignant B-lineage cells, such as chronic lymphocytic leukemia, Non-Hodgkin lymphoma, and acute lymphoblastic leukemia, comprising
(a) an anti-CD19 antibody lacking an Fc fragment,
(b) a modified exotoxin A protein having both Domains II and III, but lacking Domain I, and
(c) a peptide linker joining the C-terminal end of the antibody to the N-terminal end of the modified exotoxin A protein, said linker being substantially resistant to extracellular cleavage.

18. The immunotoxin of claim 17, wherein said modified exotoxin A protein has a C-terminal KDEL sequence (SEQ ID NO: 6) that promotes transport of the protein to the endoplasmic reticulum of cells that have taken up the immunotoxin.

19. The immunotoxin of claim 17, wherein the modified exotoxin A protein has the sequence identified by SEQ ID NO: 3.

20. The immunotoxin of claim 17, wherein said antibody is a single-chain scFv antibody composed of a variable-region light chain coupled to a variable-region heavy chain through a glycine/serine peptide linker.

21. The immunotoxin of claim 17, wherein the antibody is coupled to the modified exotoxin protein through a glycine/serine peptide linker.

22. The immunotoxin of claim 21, wherein the linker coupling the antibody to the modified exotoxin protein has the sequence identified as SEQ ID NO: 5.

23. A method of treating a subject having a cancer associated with malignant B-lineage cells, such as chronic lymphocytic leukemia, Non-Hodgkin lymphoma, and acute lymphoblastic leukemia, comprising administering to the patient, a therapeutically effective amount of an immunotoxin composed of:
(a) an anti-CD19 antibody lacking an Fc fragment,
(b) a modified exotoxin A protein having both Domains II and III, but lacking Domain I, and
(c) a peptide linker joining the C-terminal end of the antibody to the N-terminal end of the modified exotoxin A protein, said linker being substantially resistant to extracellular cleavage.

24. The method of claim 23, wherein said modified exotoxin A protein in the immunotoxin administered has a C-terminal KDEL sequence (SEQ ID NO: 6) that promotes transport of the protein to the endoplasmic reticulum within cells that have taken up the immunotoxin.

25. The method of claim 24, wherein the modified exotoxin A protein in the immunotoxin administered has the sequence identified by SEQ ID NO: 3.

26. The method of claim 23, wherein said antibody is a single-chain scFv antibody composed of a variable-region light chain coupled to a variable-region heavy chain through a glycine/serine peptide linker.

27. The method of claim 26, wherein the antibody in the immunotoxin administered is coupled to the modified exotoxin protein through a glycine/serine peptide linker.

28. The method of claim 27, wherein the linker coupling the antibody to the modified exotoxin protein in the immunotoxin administered has the sequence identified as SEQ ID NO: 5.

29. A method for treating an autoimmune disease, such as multiple sclerosis, rheumatoid arthritis, and SLE, comprising administering to the patient, a therapeutically effective amount of an immunotoxin composed of:
(a) an anti-CD19 antibody lacking an Fc fragment,
(b) a modified exotoxin A protein having both Domains II and III, but lacking Domain I, and
(c) a peptide linker joining the C-terminal end of the antibody to the N-terminal end of the modified exotoxin A protein, said linker being substantially resistant to extracellular cleavage.

30. A method for delivering exotoxin A (ETA) to a human subject, in the treatment of a cancer having cancer-specific cell-surface antigens, comprising
(a) replacing Domain I of the ETA with a single-chain antibody specific against the cell-surface antigen and a peptide linker joining the C-terminal end of the antibody to the N-terminal end of the modified ETA, said linker being substantially resistant to extracellular cleavage, and
(b) replacing the REDLK C-terminal sequence (SEQ ID NO: 7) of ETA with a KDEL sequence (SEQ ID NO: 6) that promotes transport of the protein to endplasmic reticulum.

31. The method of claim 30, for treating a subject having a cancer associated with malignant B-lineage cells, such as chronic lymphocytic leukemia, Non-Hodgkin lymphoma, and acute lymphoblastic leukemia, wherein the single-chain antibody is specific against CD19 B-cell antigen.

32. The method of claim 30, wherein said linker includes a glycine/serine peptide linker.

33. The method of claim 32, wherein said linker has the sequence identified as SEQ ID NO: 5.
